(19) United States
(12) Patent Application Publication
(10) Pub. No.: US 2003/0152558 A1
(43) Pub. Date: Aug. 14, 2003

(54) Methods and compositions for the use of stromal cells to support embryonic and adult stem cells

(76) Inventors: Christopher Luft, Chapel Hill, NC (US); William O. Wilkison, Bahama, NC (US); Bentley Cheatham, Durham, NC (US); Jeffrey M. Gimble, Chapel Hill, NC (US); Yuan-Di C. Halvorsen, Branford, CT (US)

Correspondence Address: KING & SPALDING, 191 PEACHTREE STREET, N.E., ATLANTA, GA, 30303-1763, US

(21) Appl. No.: 10/293,394

(22) Filed: Nov. 12, 2002

Related U.S. Application Data
(60) Provisional application No. 60/344,555, filed on Nov. 9, 2001.

(51) Int. Cl. A61K 48/00; C12N 5/08

(52) U.S. Cl. 424/93.21; 435/366

(57) ABSTRACT

The invention provides cells, compositions and methods based on the use of stromal cells to support the proliferation of undifferentiated embryonic or adult stem cells in vitro. The stem cells produced in the method are useful in providing a source of uncommitted or differentiated and functional cells for research, transplantation and development of tissue engineered products for the treatment of human diseases and traumatic tissue injury repair in any tissue or organ site within the body.

### CROSS REFERENCE TO RELATED APPLICATION

This application claims priority to U.S. Serial No. 60/344,555 filed Nov. 9, 2001.

### FIELD OF INVENTION

This invention provides methods and compositions for the use of stromal cells derived from adipose tissue, bone, bone marrow, cartilage, connective tissue, foreskin, ligaments, peripheral blood, placenta, smooth muscle, skeletal muscle, tendons, umbilical cord, or other sites in the isolation, culture and maintenance of embryonic or adult stem cells and uses thereof.

### BACKGROUND OF INVENTION

Embryonic stem cells are derived from the inner cell mass of blastocyst-stage embryos [Odorico et al. 2001, Stem Cells 19:193-204; Thomson et al. 1995. Proc Natl Acad Sci USA. 92:7844-7848.; Thomson et al. 1998. Science 282:1145-1147]. These cells are described variously as pluripotent and totipotent stem cells. Their distinguishing characteristic is their capacity to give rise to differentiated daughter cells representing all three germ layers of the embryo and the extra-embryonic cells that support development. Stem cells have been isolated from other sites in the embryo and in adult tissues. Pluripotent or totipotent stem cells capable of differentiating into cells reflecting all three germ layers of the embryo can be isolated from the primordial germinal ridge of the developing embryo, from teratocarcinomas, and from non-embryonic tissues, including but not limited to the bone marrow, brain, liver, pancreas, peripheral blood, placenta, skeletal muscle, and umbilical cord blood. These cells display a number of common properties. They display high levels of alkaline phosphatase enzyme activity [Shamblott et al. 1998, Proc Natl Acad Sci USA 95:13726-13731]. They also express high levels of the telomerase enzyme, a ribonucleoprotein that catalyzes the addition of telomere repeats to chromosome ends. This activity maintains the chromosome length and is correlated with cell immortality [Odorico et al 2001, Stem Cells 19:193-204].

Embryonic stem cells of human origin express cell surface markers including but not limited to stage-specific embryonic antigens 3 and 4 (SSEA-3 and SSEA-4), high molecular weight glycoproteins TRA-1-60 and RA-1-81, and alkaline phosphatase [Amit M et al. 2000, Dev Biol 227:271-278; Odorico et al, Stem Cells 19:193-204]. In their undifferentiated state, the embryonic stem cells retain their ability to express the transcription factor October 4; with differentiation commitment, the level of October 4 decreases [Reubinoff et al, 2000, Nature Biotechnology 18:399-404.; Schuldiner et al. 2000, Proc Natl Acad Sci USA 97:11307-11312].

The embryonic stem cells undergo lineage specific differentiation in response to a panel of cytokines. Representative examples from the literature are cited below but the list is not intended to be comprehensive or exhaustive. Transforming growth factor β1 and activin A inhibit endodermal and ectodermal differentiation while promoting mesodermal lineages such as skeletal and cardiac muscle [Schuldiner et al. 2000, Proc Natl Acad Sci USA 97:11307-11312]. Retinoic acid, basic fibroblast growth factor, bone morphogenetic protein 4, and epidermal growth factor induce both ectodermal (skin, brain) and mesodermal (chondrocyte, hematopoietic) lineages [Schuldiner et al. 2000]. Other factors, such as nerve growth factor and hepatic growth factor, promote differentiation along all three embryonic lineages (ectodermal, endodermal, mesodermal). Still other factors such as platelet derived growth factor promote glial cell differentiation [Brustle et al 1999, Science 285(5428):754-6]. Transplantation of the embryonic stem cells into skeletal muscle tissue or other tissue site of immunodeficient mice leads to the development of a teratocarcinoma, exhibiting the differentiation of gut-like structures, neural epithelium, cartilage, striated muscle, glomeruli and other tissue types [Thomson et al 1998, Curr Top Dev Biol 38:133-165; Amit et al. 2000, Dev Biol 227:271-278; Reubinoff et al. 2000, Nature Biotechnology 18:399-404]. Alternatively, the embryonic stem cells differentiate into cells derived from all three germinal layers when cultured in vitro as embryoid bodies [Itskovitz-Eldor J et al. 2000, Mol Med 6:88-95; Reubinoffet al. 2000, Nature Biotechnology 18:399-404].

Current methods for the isolation and maintenance of embryonic and other stem cell lines rely on the use of murine embryonic fibroblasts (MEF). Prior to co-culture, the MEF are irradiated (levels of between 35-50 gray) to reduce cell proliferation without compromising metabolic function [Shamblott et al. 1998, Proc Natl Acad Sci USA 95:13726-13731; Amit et al 2000, Dev Biol 227:271-278]. Embryonic stem cells are isolated from the inner cell mass from blastocyst stage embryos by immunosurgery [Reubinoff et al. 2000, Nature Biotechnology 18:399-404; Thomson et al. 1998, Science 282:1145-1147; Odoricoet al 2001, Stem Cells 19:193-204]. The zona pellucida is digested with pronase, the inner cell mass is isolated by immunosurgery with an anti-human serum antibody followed by exposure to guinea pig complement, and the resulting cells plated onto the irradiated MEF feeder layer culture [Reubinoff et al. 2000, Nature Biotechnology 18:399-404; Thomson et al. 1998, Science 282:1145-1147]. Alternatively, cells are isolated from the gonadal ridges and mesenteries of 5 to 9 week old post-fertilization human embryos following mechanical disaggregation and trypsin/EDTA digestion or hyaluronidase/collagenase IV/Dnase digestion and subsequent plating onto an MEF feeder layer [Shamblott et al. 1998, Proc Natl Acad Sci USA 95:13726-13731]. Cultures are maintained in the presence of Dulbecco's Modified Eagle's Medium (no pyruvate, high glucose formulation), Knock Out Dulbecco's Modified Eagle's Medium or equivalent medium supplemented with 15-20% fetal bovine serum or 15-20% Knock Out SR (Gibco/BRL, Gaithersburg Md.), a serum replacement optimized for embryonic stem cell growth [Price et al WO98/30679], 0.1 mM nonessential amino acids, 0.1 mM 2-mercaptoethanol, 2 mM glutamine, antibiotics, and the addition of up to 2,000 units/ml of human recombinant leukemia inhibitory factor (LIF), up to 4 ng/ml of human recombinant basic fibroblast growth factor, and up to 10 μM forskolin [Amit et al. 2000, Dev Biol 227:271-278; Reubinoff et al. 2000, Nature Biotechnology 18:399-404; Shamblott et al. 1998, Proc Natl Acad Sci USA 95:13726-13731; Thomson et al. 1998, Science 282:1145-1147].

The frequency of embryonic stem cell clones has been noted to increase several-fold with the use of serum replacements [Amit et al. 2000, Dev Biol 227:271-278]. The presence of basic fibroblast growth factor is required for the continued undifferentiated proliferation of the clonal embryonic stem cells. The combined presence of bFGF, LIF, and forskolin is associated with the development of tight, compacted multicellular human embryonic stem cell colonies as opposed to flattened, loose colonies seen with other primates (rhesus) [Shamblott et al. 1998, Proc Natl Acad Sci USA 95:13726-13731]. After 9 to 15 days, individual colonies are dissociated into clumps by exposure to calcium and magnesium free phosphate buffered saline with 1 mM EDTA or other divalent cation chelator, by exposure to dispase (10 mg/ml), or by mechanical dissociation with a micropipette [Thomson J A, Itskovitz-Eldor J, Shapiro S S, et al. 1998. Embryonic stem cell lines derived from human blastocysts. Science 282:1145-1147]. The resulting cells or cell clumps are sequentially plated onto irradiated mouse embryonic fibroblasts in fresh medium [Thomson J A et al. 1998, Science 282:1145-1147; Reubinoff et al. 2000, Nature Biotechnology 18:399-404]. Clones are expanded on the order of every 7 days and display a doubling time of approximately 36 hours [Amit et al. 2000, Dev Biol 227:271-278]. Subsequent passage of the clones is carried out by repeating the cell disruption procedure (digestion, micropipette manipulation) and by plating the resulting cells on irradiated MEFs [Amit et al. 2000, Dev Biol 227:271-278].

The current methods for the isolation, culture and expansion of human embryonic stem cells are limited by their reliance on a murine embryonic fibroblast feeder layer. The fact that such murine MEFs were used to isolate the existing 60 human stem cell clones presents a hurdle to the use of these cells in clinical therapies [Gillis J, Connolly C. Aug. 24, 2001. “Taint of mouse cells might hinder stem researchers”. Washington Post]. While investigators have likely begun to explore the use of cytokine and extracellular matrix supplements to avoid the use of a murine embryonic fibroblast feeder layer in the growth of human embryonic stem cells and stem cells from other tissues and donor sites, such studies are in their infancy [Carpenter et al, Exp Neurol 158:265-278; Odorico 2001, Stem Cells 19:193-204]. However, International patent WO 01/51616 to Schiff discloses a method for culturing human pluripotent stem cells in the absence of feeder cells, such as MEFs. It remains to be demonstrated that a totipotent stem cell can be maintained indefinitely in an undifferentiated state in the absence of feeder cells [Odorico 2001, Stem Cells 19:193-204].

Therefore, the object of the current invention is to provide a method and compositions to assist in the isolation, culture and maintenance of stem cells.

### SUMMARY OF THE INVENTION

The present invention provides methods and compositions that include the use of tissue-derived stromal cells, including adipose-derived stromal cells, as a feeder layer in the isolation, culture, and maintenance of adult, embryonic and other stem cells. Methods and compositions for consistent support of stem cells by irradiated adipose-derived stromal cells are provided.

In one aspect of the invention, isolated tissue-derived cells are supplemented with additional growth factors, cytokines, and chemokines to isolate, culture and maintain stem cells.

In another aspect of this invention, the isolated tissue-derived cells, including adipose-derived tissue cells, are irradiated before the culture media is supplemented with additional growth factors, cytokines, and/or chemokines. Alternatively, the isolated tissue-derived cells are irradiated after the culture media is supplemented with additional growth factors, cytokines, and/or chemokines.

In still another aspect of the present invention, tissue-derived stromal cells are genetically engineered to express one or more proteins or growth factors that facilitate the culture and maintenance of stem cells. Alternatively, the tissue-derived stromal cells are irradiated after being genetically engineered to express such proteins or growth factor. Such factors are used to maintain the stems cells in an undifferentiated state or alternatively to direct their differentiation.

According to the details of the invention described herein, the tissue-derived stromal cells, including adipose-derived stromal cells, are used to culture and maintain embryonic stem cells. Irradiated tissue-derived stromal cells are also used to culture and maintain embryonic stem cells.

In still another aspect of the invention, tissue-derived stromal cells, including adipose-derived stromal cells, are used in the culture and maintenance of stem cells of various types, including but not limited to, neuronal stem cells, liver stem cells, hematopoietic stem cells, umbilical cord blood stem cells, epidermal stem cells, gastrointestinal stem cells, endothelial stem cells, muscle stem cells, mesenchymal stem cells and pancreatic stem cells. Irradiated tissue-derived stromal cells are also used to culture and maintain such stem cells.

In another aspect of the invention, isolated tissue-derived stromal cells, including adipose-derived stromal cells, are supplemented with growth factors, cytokines and chemokines that are used alternately to enhance proliferation, to maintain, and to facilitate the directed differentiation of co-cultured stem cells. Alternatively, the isolated tissue-derived stromal cells are first irradiated and then supplemented with growth factors, cytokines and chemokines which are used alternately to enhance proliferation, to maintain, and to facilitate the directed differentiation of co-cultured stem cells.

Other objects and features of the invention will be more fully apparent from the following disclosure and appended claims.

### BRIEF DESCRIPTION OF THE DRAWINGS

FIG. 1 is a representative flow cytometric analysis of human adipose-derived stromal cells. Undifferentiated stromal cells isolated from a single donor were stained with monoclonal antibodies against indicated antigens (solid line, right of each panel) or isotype monoclonal control antibody (dotted line, left of each panel). Representative n=5 donors. Bar indicates fluorescent intensity >99% control. The adipose derived stromal cells express a number of adhesion and surface proteins. Many of these proteins have the potential to serve a hematopoietic supportive function and all of them are shared in common by bone marrow stromal cells.

FIG. 2 shows a PCR analysis of lipopolysaccharide (LPS) induction of cytokine mRNA. Adipose derived stromal cells were induced with 100 ng/ml LPS for 0 or 4 hours and harvested for total RNA. Reverse transcribed cDNAs were amplified with specific primer sets for interleukins 6 and 8, granulocyte-, macrophage-, and granulocyte/macrophage-colony stimulating factors, flt-3 ligand, and leukemia inhibitory factor. Actin signal served as a control for equivalent cDNA levels in each reaction. PCR products were sequence confirmed. In common with both murine and human bone marrow-derived stromal cells, adipose derived stromal cells expressed the following cytokine mRNAs: interleukins 6, 7, 8, and 11 (IL-6,-7,-8,-11), leukemia inhibitory factor (LIF), macrophage-colony stimulating factor (M-CSF), granulocyte-macrophage-colony stimulating factor (GM-CSF), granulocyte-colony stimulating factor (G-CSF), flt-3 ligand, stem cell factor, tumor necrosis factor α (TNFα) and bone morphogenetic proteins 2 and 4 t (BMP-2, -4).

FIG. 3 shows data for total cell expansion for various co-cultures. Hematopoietic cells from 12-day adipose stroma co-cultures were examined for total cell expansion (left panel), CD34+ cell expansion (middle panel) or seeded on MS5 cells for 5 weeks and the expansion of myeloid long term culture initiating (LTC) cells. In the absence of exogenous cytokines, adipose-derived stromal cells supported a 5.1-fold expansion of total hematopoietic cell numbers (average, n=4 stromal donors, n=2 UCB donors; range 2-9.4). This corresponded to a 2.4-fold expansion of the CD34⁺ UCB cell population (average, n=4 stromal donors, n=2 UCB donors; range 1.4-3.3).

### DETAILED DESCRIPTION OF THE INVENTION

The present invention provides methods and composition that include the use of tissue-derived stromal cells, including adipose-derived stromal cells, as a feeder layer in the isolation, culture, and maintenance of adult, embryonic and other stem cells. In one embodiment of the present invention, methods and compositions for consistent support of stem cells by irradiated stromal cells derived from subcutaneous, mammary, gonadal, omental or other adipose tissue sites is provided.

In another embodiment of the invention, isolated tissue-derived cells, including adipose-derived stromal cells, are supplemented with additional growth factors, cytokines, and chemokines, including but not limited to, leukemia inhibitory factor, IL-1 through IL-13, IL-15 through IL-17, IL-19 through IL-22, granulocyte macrophage colony stimulating factor (GM-CSF), granulocyte colony stimulating factor (G-CSF), macrophage colony stimulating factor (M-CSF), erythropoietin (Epo), thrombopoietin (Tpo), Flt3-ligand, BAFF (novel ligand of TNF family for B cell activating factor), artemin (a neurotrophic factor belonging to the GDNF family), bone morphogenic protein factors, epidermal growth factor (EGF), glial derived neurotrophic factor, lymphotactin, macrophage inflamamatory proteins (alpha and beta), myostatin (also known as Growth Differentiation Factor-8), neurturin, nerve growth factors, platelet derived growth factors, placental growth factor, pleiotrophin, stem cell factor, stem cell growth factors, transforming growth factors, tumor necrosis factors, Vascular Endothelial Cell Growth Factors, and fibroblast growth factors including FGF-4 through FGF-10, FGF-16 through FGF-20, FGF-acidic and basic fibroblast growth factor, to isolate, culture and maintain stem cells. In another embodiment of this invention the isolated tissue-derived cells are irradiated before the culture media is supplemented with such additional growth factors, cytokines, and/or chemokines. Alternatively, the isolated tissue-derived cells are irradiated after the culture media is supplemented with additional growth factors, cytokines, and/or chemokines.

In a further embodiment of the present invention, tissue-derived stromal cells, including adipose-derived stromal cells, are genetically engineered to express proteins, including but not limited to, leukemia inhibitory factor, IL-1 through IL-13, IL-15 through IL-17, IL-19 through IL-22, granulocyte macrophage colony stimulating factor (GM-CSF), granulocyte colony stimulating factor (G-CSF), macrophage colony stimulating factor (M-CSF), erythropoietin (Epo), thrombopoietin (Tpo), Flt3-ligand, BAFF (novel ligand of TNF family for B cell activating factor), artemin (a neurotrophic factor belonging to the GDNF family), bone morphogenic protein factors, epidermal growth factor (EGF), glial derived neurotrophic factor, lymphotactin, macrophage inflamamatory proteins (alpha and beta), myostatin (also known as Growth Differentiation Factor-8), neurturin, nerve growth factors, platelet derived growth factors, placental growth factor, pleiotrophin, stem cell factor, stem cell growth factors, transforming growth factors, tumor necrosis factors, Vascular Endothelial Cell Growth Factors, and fibroblast growth factors including FGF-4 through FGF-10, FGF-16 through FGF-20, FGF-acidic and basic fibroblast growth factor, to isolate, culture and maintain stem cells. In an alternative embodiment, the tissue-derived stromal cells are irradiated after being so genetically engineered. In still another modification of this embodiment, such engineered cells are used to direct the differentiation of the stem cells. Alternatively, the engineered cells are used to maintain the stem cells in an undifferentiated state.

In another embodiment of the present invention, tissue-derived stromal cells, including adipose-derived stromal cells, are used to culture and maintain embryonic stem cells. In an alternative embodiment, irradiated tissue-derived stromal cells are used to culture and maintain embryonic stem cells.

In still another embodiment of the present invention, tissue-derived stromal cells, including adipose-derived stromal cells, are used to isolate, culture, and maintain stem cells originating from adult tissues, including but not limited to, neuronal stem cells, liver stem cells, hematopoietic stem cells, epidermal stem cells, gastrointestinal stem cells, endothelial stem cells, muscle stem cells, mesenchymal stem cells and pancreatic stem cells. In an alternative embodiment, the tissue-derived stromal cells are irradiated.

In another embodiment of the present invention, isolated tissue-derived stromal cells, including adipose-derived stromal cells, are supplemented with growth factors, cytokines and chemokines that are used alternately to enhance proliferation, to maintain, and to facilitate the directed differentiation of co-cultured stem cells. Alternatively, the isolated tissue-derived stromal cells are first irradiated and then supplemented with such growth factors, cytokines and chemokines.

Cells with features similar to adipose tissue-derived stromal cells are obtained from other tissue sites. These include, but are not limited to, bone, bone marrow, cartilage, connective tissue, foreskin, ligaments, peripheral blood, placenta, skeletal muscle, smooth muscle, tendons, and umbilical cord blood [see U.S. Pat. No. 5,226914 to Caplan and Haynesworth; Erices et al 2000, Br. J. Haematol. 109: 235-242; Gimble 1990, New Biologist 2: 304-312; Gimble et al 1996, Bone 19: 421-428]. While none of the stromal cells derived from any or all of these tissues are identical, it is probable that they share sufficient features in common to allow them to serve similar functions in vitro. In particular, stromal cells obtained from these diverse tissue sites can serve as a feeder layer to support either embryonic or adult stem cell proliferation and maintenance in an undifferentiated state. Furthermore, based on the unique features of each of these types of stromal cells, they may each be optimal for the growth and maintenance of specific types of stem cells.

I. Definitions

“Embryonic Stem Cells” is intended as any primitive (undifferentiated) cells derived from the embryo (inner cell mass of the blastocyst) that have the potential to become a wide variety of specialized cells. Embryonic stem cells are capable of undergoing an unlimited number of symmetrical divisions without differentiating (long-term self-renewal). They also exhibit and maintain a stable, full (diploid), normal complement of chromosomes. The cells express high levels of telomerase activity and are distinguished by specific cell surface proteins and transcription factors.

“Adult Stem Cells” is intended as any undifferentiated cell found in a differentiated post-embryonic tissue that can renew itself and (with certain limitations) differentiate to yield all the specialized cell types of the tissue from which it originated and into a wide variety of other cell types.

“Leukemia Inhibitory Factor” (LIF) is intended to mean a 22 kDa protein member of the interleukin-6 cytokine family that has numerous biological functions. LIF has the capacity to induce terminal differentiation in leukemic cells, induce hematopoietic differentiation in normal and myeloid leukemia cells, induce neuronal cell differentiation, and stimulate acute-phase protein synthesis in hepatocytes. LIF has also been shown to be necessary for maintaining embryonic stem cells in a proliferative, undifferentiated state.

“Feeder Layer” is intended to mean cells that have been inactivated by chemical or radiologic means so they will not divide yet will still produce the growth factors, cytokines and other cell-derived products necessary in co-culture to maintain undifferentiated, pluripotent stem cells. Historically, mouse embryonic fibroblasts have been used as a feeder layer in the support of embryonic stem cells.

“Fibroblast Growth Factor-basic” (FGF-b) is a 17.2 kDa protein that is a heparin binding growth factor that stimulates the proliferation of a wide variety of cells including mesenchymal, neuroectodermal and endothelial cells. Human FGF-b is a potent hematopoietic cytokine and exerts a powerful angiogenic activity in vivo. FGF-b also antagonizes cytokine-mediated differentiation of a human leukemic cell line. Hence, bFGF could promote proliferation of progenitor cells by antagonizing their differentiation.

A “pluripotential embryonic stem cell” is a cell that can give rise to many differentiated cell types in an embryo or adult, including the germ cells (sperm and eggs). Pluripotent embryonic stem cells are also capable of self-renewal. Thus, these cells not only populate the germ line and give rise to a plurality of terminally differentiated cells that comprise the adult specialized organs, but also are able to regenerate themselves.

The term “transgenic” is used to describe any animal or any part thereof, including but not restricted to, cells, cultures or tissues that includes exogenous genetic material within its cells. Cells of the invention can have the DNA added to them and these cells can then be used for transplantation or for in vitro production of hormones, cells or tissues.

“Transgene” means any piece of DNA inserted by artifice into a cells that becomes part of the genome of the cell, cell line, tissue or organism (i.e. either stably integrated or as a stable extrachromosomal element) which develops from that cell. Such a transgene may include a gene that is partly or entirely heterologous or foreign to the cell or organism to which the heterologus gene is introduced, or may represent a gene homologous to an endogenous gene of the organists. Included within the definition is a transgene created by the providing of an RNA sequence that is transcribed into DNA and then incorporated into the genome. The term “transgenic” additionally includes any organisms or in part thereof, including, but not limited to, cells, cell lines, cell cultures or tissues whose genome has been altered by in vitro manipulation or by any transgenic technology.

“Transforming Growth Factor β” (TGFβ) is a 55 kDa, 391 amino acid (aa) pre-proprotein that consists of a 23 aa signal sequence, a 256 aa pro-region and a 112 aa mature segment. Prior to secretion, the pro-region is cleaved at an RxxR site with a furin-like protease. This generates a non-glycosylated, 25 kDa, disulfide-linked mature dimer that non-covalently associates with its previously attached disulfide-linked pro-regions to form a “latent complex”. This complex is secreted. Activation occurs extracellularly under a variety of conditions most likely via a transmembrane serine/threonine kinase to initiate an intracellular signal cascade mediated by the Smad family of transcription factors.

“Basic Fibroblast Growth Factor” (bFGF), also known as FGF-2, is an 18 kDa, non-glycosylated polypeptide that shows both intracellular and extracellular activity. BFGF is secreted as a monomer. Following secretion, bFGF is sequestered on either cell surface heparin sulfate (HS) or matrix glycosaminoglycans. Although bFGF is secreted as a monomer, cell surface HS seems to dimerize monomeric bFGF in a non-covalent side-to-side configuration that is subsequently capable of dimerizing and activating FGF receptors.

“Platelet Derived Growth Factor” (PDGF) is a 30 kDa homo- or heterodimeric combination of two genetically distinct, but structurally related, polypeptide chains designated A and B. It was originally identified as a platelet derived fibroblast mitogen in serum. Subsequent studies have demonstrated that many cell types secrete PDGF and that the cytokine is a mitogen for cells of the mesodermal lineage (muscle, bone, connective tissue).

II. Isolation of Adipose-Derived Stem Cells

Adipose tissue offers a source of multipotential stromal cells. Adipose tissue is readily accessible and abundant in many individuals. Obesity is a condition of epidemic proportions in the United States, where over 50% of adults exceed the recommended BMI based on their height. Adipocytes can be harvested by liposuction on an outpatient basis. This is a relatively non-invasive procedure with cosmetic effects that are acceptable to the vast majority of patients. It is well documented that adipocytes are a replenishable cell population. Even after surgical removal by liposuction or other procedures, it is common to see a recurrence of adipocytes in an individual over time. This suggests that adipose tissue contains stromal stem cells that are capable of self-renewal.

“Adipose tissue-derived stromal cells” are obtained from minced human adipose tissue by collagenase digestion and differential centrifugation [Halvorsen et al, 2001, Tissue Eng. 7(6):729-41; Hauner et al, 1989, J Clin Invest 84:1663-1670; Rodbell 1966, J Biol Chem 241:130-139]. It has been demonstrated that human adipose tissue-derived stromal cells can differentiate along the adipocyte, chondrocyte, and osteoblast lineage pathways [Erickson et al, 2002, Biochem Biophys Res Commun 290(2):763-9; Gronthos et al 2001, Journal of Cell Physiology 89(1):54-63; Halvorsen et al, 2001, Metabolism 50:407-413; Harp et al, 2001, Biochem Biophys Res Commun 281:907-912; Saladin et al, 1999, Cell Growth & Diff 10:43-48; Sen et al, 2001, Journal of Cellular Biochemistry 81:312-319; Zhou et al, 1999, Biotechnol Techniq 13:513-517; Zuk et al, 2001, Tissue Eng 7:211-28].

Adipose tissue offers many practical advantages for tissue engineering applications. First, it is abundant. Second, it is accessible to harvest methods with minimal risk to the patient. Third, it is replenishable. While stromal cells represent less than 0.01% of the bone marrow's nucleated cell population, there are up to 8.6×10⁴ stromal cells per gram of adipose tissue [Sen et al, 2001, Journal of Cellular Biochemistry 81:312-319]. Ex vivo expansion over 2 to 4 weeks yields up to 500 million stromal cells from 0.5 kilograms of adipose tissue. These cells can be used immediately or cryopreserved for future autologous or allogeneic applications.

The adipose derived stromal cells express a number of adhesion and surface proteins, including, but not limited to, the following cell surface markers: CD29 (β1 integrin), CD44 (hyaluronate receptor), CD49_{d} (α4 integrin), CD54-ICAM1 CD105-Endoglin; CD106-VCAM-1 CD166-ALCAM; and the following cytokines: Interleukins 6, 7, 8, 11 Macrophage-Colony Stimulating Factor, GM-Colony Stimulating Factor, Granulocyte-Colony Stimulating Factor, Leukemia Inhibitory Factor (LIF), Stem Cell Factor, and Bone Morphogenetic. Many of these proteins have the potential to serve a hematopoietic supportive function and all of them are shared in common by bone marrow stromal cells.

Cells with features similar to adipose tissue-derived stromal cells can be obtained from other tissue sites. These include, but are not limited to, bone, bone marrow, cartilage, connective tissue, foreskin, ligaments, peripheral blood, placenta, skeletal muscle, smooth muscle, tendons, and umbilical cord blood [U.S. Pat. No. 5,226,914 to Caplan and Haynesworth; Erices et al, 2000, Br J Haematol. 109:235-42; Gimble J M 1990, The New Biologist 2:304-312; Gimble et al, 1996, Bone 19:421-428]. While none of the stromal cells derived from any or all of these tissues are identical, it is probable that they share sufficient features in common to allow them to serve similar functions in vitro. In particular, stromal cells obtained from these diverse tissue sites will also be able to serve as a feeder layer to support either embryonic or adult stem cell proliferation and maintenance in an undifferentiated state.

WO 00/53795 to the University of Pittsburgh and The Regents of the University of California discloses adipose derived stem cells that can be grown and expanded to provide hormones and conditioned media for supporting the growth and expansion of other cell populations, which further can be genetically modified to repress or express certain genes. In one example, human lipo-derived stem cells were co-cultured with hematopoetic stem cells from umbilical cord blood. Over a two-week period, human adipose derived stromal cells maintained the survival and supported the growth of human hematopoetic stem cells, thereby illustrating the utility of such a system in maintaining stem cell growth.

U.S. Pat. No. 5,922,597 to Verfaillie discloses methods directed to utilizing stromal cells to provide conditioned media to support the growth and maintenance of stem cells. It is taught that stromal cells and stem cells may be combined in a co-culture.

The adipose tissue derive stromal cells useful in the methods of invention are isolated by a variety of methods known to those skilled in the art such as described in WO 00/53795 to the University of Pittsburgh et al. In a preferred method, adipose tissue is isolated from a mammalian subject, preferably a human subject. A preferred source of adipose tissue is omental adipose. In humans, the adipose is typically isolated by liposuction. If the cells of the invention are to be transplanted into a human subject, it is preferable that the adipose tissue be isolated from that same subject so as to provide for an autologous transplant. Alternatively, the transplanted tissue may be allogenic.

As a non-limiting example, in one method of isolating adipose tissue derived stromal cells, the adipose tissue is treated with collagenase at concentrations between 0.01 to 0.5%, preferably 0.04 to 0.2%, most preferably 0.1%, trypsin at concentrations between 0.01 to 0.5%, preferably 0.04 to 0.04%, most preferably 0.2%, at temperatures between 25° to 50° C., preferably between 33° to 40° C., most preferably at 37° C., for periods of between 10 minutes to 3 hours, preferably between 30 minutes to 1 hour, most preferably 45 minutes. The cells are passed through a nylon or cheesecloth mesh filter of between 20 microns to 800 microns, more preferably between 40 to 400 microns, most preferably 70 microns. The cells are then subjected to differential centrifugation directly in media or over a Ficoll or Percoll or other particulate gradient. Cells are centrifuged at speeds of between 100 to 3000×g, more preferably 200 to 1500×g, most preferably at 500×g for periods of between 1 minute to 1 hour, more preferably 2 to 15 minutes, most preferably 5 minutes, at temperatures of between 4° to 50° C., preferably between 20° to 40° C., most preferably at 25° C.

In yet another method of isolating adipose-derived stromal cells a mechanical system such as described in U.S. Pat. No. 5,786,207 to Katz et al is used. A system is employed for introducing an adipose tissue sample into an automated device, subjecting it to a washing phase and a dissociating phase wherein the tissue is agitated and rotated such that the resulting cell suspension is collected into a centrifuge-ready receptacle. In such a way, the adipose-derived cells are isolated from a tissue sample, preserving the cellular integrity of the desired cells.

Adipose-derived cells are cultured by methods disclosed in U.S. Pat. No. 6,153,432 (herein incorporated by reference). Similar techniques to isolate stromal cells from other tissues, including, but not limited to stromal cells derived from bone, bone marrow, cartilage, connective tissue, foreskin, ligaments, peripheral blood, placenta, smooth muscle, skeletal muscle, tendons, umbilical cord, or other sites, will be apparent to one skilled in the art.

III. Irradiation of Adipose-Derived Stromal Cells

The adipose tissue-derived stromal cells may be irradiated. The cells must be irradiated at a dose that inhibits proliferation, but permits the synthesis of important factors that support the embryonic stem cells. The details of such protocols are well know to those skilled in the art.

IV. Media Enhancement

It is further recognized that additional components may be added to the culture medium. Such components include antibiotics, albumin, amino acids, and other components known to the art for the culture of cells.

Other additions to the media may include IL-1 through IL-13, IL-15 through IL-17, IL-19 through IL-22, granulocyte macrophage colony stimulating factor (GM-CSF), granulocyte colony stimulating factor (G-CSF), macrophage colony stimulating factor (M-CSF), erythropoietin (Epo), thrombopoietin (Tpo), Flt3-ligand, BAFF (novel ligand of TNF family for B cell activating factor), artemin (a neurotrophic factor belonging to the GDNF family), bone morphogenic protein factors, epidermal growth factor (EGF), glial derived neurotrophic factor, lymphotactin, macrophage inflamamatory proteins (alpha and beta), myostatin (also known as Growth Differentiation Factor-8), neurturin, nerve growth factors, platelet derived growth factors, placental growth factor, pleiotrophin, stem cell factor, stem cell growth factors, transforming growth factors, tumor necrosis factors, Vascular Endothelial Cell Growth Factors, and fibroblast growth factors including FGF-4 through FGF-10, FGF-16 through FGF-20, FGF-acidic, FGF-basic, LIF and other growth factors, cytokines and chemokines which are well known in the art of cell culture and which are used alternately to enhance proliferation, to maintain, and to facilitate the directed differentiation of stem cells. Growth and proliferation enhancing amounts can vary depending on the species or strain of the cells, and type or purity of the factors. Generally, 0.5 to 500 ng/ml of each factor within the culture solution is adequate. In a more narrow range, the amount is between 10 to 20 ng/ml for FGFb and LIF. Regardless of whether the actual amounts are known, the optimal concentration of each factor can be routinely determined by one skilled in the art. Such determination is performed by titrating the factors individually and in combination until optimal growth is obtained. Additionally, other factors can also be tested to determine their ability to enhance the effect of FGFb and LIF on ES cell proliferation. As described below, such other factors, or combinations of factors when used to enhance ES cell proliferation are included within the above compositions. Also, compounds and fragments of FGFb and LIF which mimic the function of these factors are used to enhance the growth and proliferation of the cells to become ES cells and are included within the scope of the invention.

Alternatively, FGFb and LIF are used to maintain ES cells. The amounts of FGFb and LIF necessary to maintain ES cells are much less than that required to enhance growth or proliferation to become ES cells. However, the cells may be maintained on a feeder layer without the addition of growth factors. Optimally, LIF is added to enhance maintenance.

In general, FGFb or LIF from a species different from the source of the ES, primordial germ cell, germ cell or embryonic ectoderm cell are utilized. However, all the factors utilized and especially the SF utilized are preferably from the same species as the utilized cell type. However, FGFb or LIF from various species are routinely screened and selected for efficacy with a cell from a different species. Recombinant fragments of FGFb or LIF can also be screened for efficacy as well as organic compounds derived from, for example, chemical libraries.

The invention also provides a method of making a pluripotential ES cell comprising administering a growth enhancing amount of FGFb, LIF, and/or embryonic ectoderm cells under cell growth conditions, thereby making a pluripotential ES cell. Thus, primordial germ cells and embryonic ectoderm cells are cultured as a composition in the presence of these factors to produce pluripotent ES cells. As noted above, typically the composition includes a feeder layer of adipose tissue-derived stromal cells.

V. Genetic Modification of the Tissue-Derived Stromal Cells

Another aspect of the present invention relates to the introduction of foreign genes into the tissue-derived stromal cells such that the tissue-derived stromal cells carry the new genetic material and can express the desired gene product. Examples of genetic material for transduction into adipose tissue-derived stromal cells includes those which express any gene products which have a role in the growth and proliferation of the particular stem cells supported by the feeder layer.

Thus, the tissue-derived stromal cells are modified with genetic material of interest (transduced or transformed or transfected). These modified cells can then be co-cultured with embryonic or adult stem cells to allow for their proliferation.

The tissue-derived stromal cells may be genetically modified by incorporation of genetic material into the cells, for example using recombinant expression vectors.

As used herein “recombinant expression vector” refers to a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in eukaryotic expression systems preferably include a leader sequence enabling extracellular secretion of translated protein by a host cell. Alternatively, where recombinant protein is expressed without a leader or transport sequence, it may include an N-terminal methionine residue. This residue may or may not be subsequently cleaved from the expressed recombinant protein to provide a final product.

The tissue-derived stromal cells thus may have integrated a recombinant transcriptional unit into chromosomal DNA or carry the recombinant transcriptional unit as a component of a resident plasmid. Cells may be engineered with a polynucleotide (DNA or RNA) encoding a polypeptide ex vivo, for example. Cells may be engineered by procedures known in the art by use of a retroviral particle containing RNA encoding a polypeptide.

Retroviruses from which the retroviral plasmid vectors described herein are derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus. In one embodiment, the retroviral plasmid vector is MGIN, derived from murine embryonic stem cells.

The nucleic acid sequence encoding the polypeptide is under the control of a suitable promoter. Suitable promoters which may be employed include, but are not limited to, TRAP promoter, adenoviral promoters, such as the adenoviral major late promoter; the cytomegalovirus (CMV) promoter; the respiratory syncytial virus (RSV) promoter; the Rous Sarcoma promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; ITRs; the .beta.-actin promoter; and human growth hormone promoters. The promoter also may be the native promoter that controls the gene encoding the polypeptide. These vectors also make it possible to regulate the production of the polypeptide by the engineered progenitor cells. The selection of a suitable promoter will be apparent to those skilled in the art.

Vehicles other than retroviruses may be used to genetically engineer or modify the tissue-derived stromal cells. Genetic information of interest is introduced by means of any virus that can express the new genetic material in such cells. For example, SV40, herpes virus, adenovirus, adeno-associated virus and human papillomavirus are used for this purpose. Other methods can also be used for introducing cloned eukaryotic DNAs into cultured mammalian cells, for example, the genetic material to be transferred to stem cells may be in the form of viral nucleic acids.

In addition, the expression vectors may contain one or more selectable marker genes to provide a phenotypic trait for selection of transformed cells such as dihydrofolate reductase or neomycin resistance.

The tissue-derived stromal cells may be transfected through other means known in the art. Such means include, but are not limited to transfection mediated by calcium phosphate or DEAE-dextran; transfection mediated by the polycation Polybrene; protoplast fusion; electroporation; liposomes, either through encapsulation of DNA or RNA within liposomes, followed by fusion of the liposomes with the cell membrane or, DNA coated with a synthetic cationic lipid are introduced into cells by fusion.

The present invention further makes it possible to genetically engineer tissue-derived stromal cells in such a manner that they produce, in vitro or in vivo, polypeptides, hormones and proteins not normally produced in the native tissue-derived stromal cells in biologically significant amounts or produced in small amounts. These products would then be secreted into the surrounding media or purified from the cells. The tissue-derived stromal cells formed in this way can serve as continuous short term or long term production systems of the expressed substance. These genes can express, for example, hormones, growth factors, matrix proteins, cell membrane proteins, cytokines, and/or adhesion molecules.

The present invention now will be described more fully by the following examples. This invention may, however, be embodied in many different forms and should not be construed as limited to the embodiments set forth herein; rather, these embodiments are provided so that this disclosure will be thorough and complete, and will fully convey the scope of the invention to those skilled in the art.

### EXAMPLES

### Example 1

### In Vitro Support of Embryonic Stem Cells by Irradiated Human Adipose-Derived Stromal Cells

Irradiation of the adipose tissue-derived stromal cells allows them to be used to support the proliferation of human embryonic stem cells in vitro. Studies are performed in the absence or presence of exogenous growth factors, including but not limited to, leukemia inhibitory factor, IL-1 through IL-13, IL-15 through IL-17, IL-19 through IL-22, granulocyte macrophage colony stimulating factor (GM-CSF), granulocyte colony stimulating factor (G-CSF), macrophage colony stimulating factor (M-CSF), erythropoietin (Epo), thrombopoietin (Tpo), Flt3-ligand, BAFF (novel ligand of TNF family for B cell activating factor), artemin (a neurotrophic factor belonging to the GDNF family), bone morphogenic protein factors, epidermal growth factor (EGF), glial derived neurotrophic factor, lymphotactin, macrophage inflamamatory proteins (alpha and beta), myostatin (also known as Growth Differentiation Factor-8), neurturin, nerve growth factors, platelet derived growth factors, placental growth factor, pleiotrophin, stem cell factor, stem cell growth factors, transforming growth factors, tumor necrosis factors, Vascular Endothelial Cell Growth Factors, and fibroblast growth factors including FGF-4 through FGF-10, FGF-16 through FGF-20, FGF-acidic and basic fibroblast growth factor. Measures of the embryonic stem cell function are also performed.

Prior to any experiment, adipose tissue-derived stromal cells are plated at a density of between 10³ to 10⁵ cells per cm² in stromal medium [Halvorsen et al, 2001, Metabolism 50:407-413]. The cells are maintained in culture until confluent, at which time they are mitotically inactivated with 3500 to 5000 rads (1 rad=0.01 Gray) gamma irradiation. Embryonic stem cells are isolated from the inner cell mass from blastocyst stage embryos by immunosurgery according to established methods [Reubinoff et al. 2000, Nature Biotechnology 18:399-404; Thomson et al. 1998, Science 282:1145-1147; Odorico et al, 2001, Stem Cells 19:193-204]. The zona pellucida is digested with pronase and the inner cell mass is isolated by immunosurgery with an appropriate anti-species specific serum antibody followed by exposure to guinea pig complement. The resulting ICM cells are plated onto the irradiated layer of adipose tissue-derived stromal cells.

Cultures are maintained in the presence of Dulbecco's Modified Eagle's Medium (no pyruvate, high glucose formulation), Knock Out Dulbecco's Modified Eagle's Medium or equivalent medium supplemented with 15-20% fetal bovine serum or 15-20% Knock Out SR (Gibco/BRL, Gaithersburg Md.), a serum replacement optimized for embryonic stem cell growth [Price et al 1998, WO98/30679], 0.1 mM nonessential amino acids, 0.1 mM 2-mercaptoethanol, 2 mM glutamine, antibiotics, and the addition of up to 2,000 units/ml of human recombinant leukemia inhibitory factor (LIF), up to 4 ng/ml of human recombinant basic fibroblast growth factor, and up to 10 μM forskolin [Amit et al. 2000, Dev Biol 227:271-278; Reubinoff et al. 2000, Nature Biotechnology 18:399-404; Shamblott et al., 1998, Proc Natl Acad Sci USA 95:13726-13731; Thomson et al. 1998, Science 282:1145-1147].

After 9 to 15 days, individual colonies are dissociated into clumps by exposure to calcium and magnesium free phosphate buffered saline with 1 mM EDTA or other divalent cation chelator, by exposure to dispase (10 mg/ml), or by mechanical dissociation with a micropiptte [Thomson et al. 1998, Science 282:1145-1147].

The resulting cells or cell clumps are sequentially plated onto irradiated human adipose tissue-derived stromal cells in fresh medium. Clones are expanded and passaged on the order of every 7 days and display a doubling time of approximately 36 hours. Subsequent passage of the clones is carried out by repeating the cell disruption procedure (digestion, micropipette manipulation) and by plating the resulting cells on irradiated MEFs.

The maintenance of the embryonic stem cell is assessed by implantation of the putative cells into the skeletal muscle of an immunodeficient mouse. The subsequent growth of a teratocarcinoma, displaying the presence of tissues derived from all three germinal layers, provides functional evidence of the proliferation of a pluripotent stem cell by the adipose tissue-derived stromal layer.

### Example 2

### In Vitro Support of Human Embryonic Stem Cell Lines by Irradiated Human Adipose-Derived Stromal Cells

Prior to any experiment, human adipose tissue-derived stromal cells are plated at a density of between 10³ to 10⁵ cells per cm² in stromal medium [Halvorsen et al, 2001, Tissue Eng. 7(6):729-41] as in Example 1. The cells are maintained in culture until confluent, at which time they are mitotically inactivated with 3500 to 5000 rads (1 rad=0.01 Gray) gamma irradiation. Existing human embryonic stem cell lines are dissociated from a murine embryonic fibroblast feeder layer by exposure to calcium and magnesium free phosphate buffered saline with 1 mM EDTA or other divalent cation chelator, by exposure to dispase (10 mg/ml), or by mechanical dissociation with a micropipette [Thomson et al. 1998, Science 282:1145-1147]. The resulting individual cells and cell clumps are plated onto the established irradiated human adipose tissue-derived stromal cell feeder layer. Cultures are maintained in the presence of Dulbecco's Modified Eagle's Medium (no pyruvate, high glucose formulation), Knock Out Dulbecco's Modified Eagle's Medium or equivalent medium supplemented with 15-20% fetal bovine serum or 15-20% Knock Out SR (Gibco/BRL, Gaithersburg Md.), a serum replacement optimized for embryonic stem cell growth [Price et al, WO98/30679], 0.1 mM nonessential amino acids, 0.1 mM 2-mercaptoethanol, 2 mM glutamine, antibiotics, and the addition of up to 2,000 units/ml of human recombinant leukemia inhibitory factor (LIF), up to 4 ng/ml of human recombinant basic fibroblast growth factor, and up to 10 μM forskolin [Amit et al. 2000, Dev Biol 227:271-278; Reubinoff et al. 2000, Nature Biotechnology 18:399-404; Shamblott et al. 1998, Proc Natl Acad Sci USA 95:13726-13731; Thomson et al, 1998, Curr Top Dev Biol 38:133-165].

Clones are expanded and passaged on the order of every 7 days and display a doubling time of approximately 36 hours [Amit et al. 2000, Dev Biol 227:271-278]. Subsequent passage of the clones is carried out by repeating the cell disruption procedure (digestion, micropipette manipulation) and by plating the resulting cells on irradiated MEFs.

The maintenance of the embryonic stem cell is assessed by implantation of the putative cells into the skeletal muscle of an immunodeficient mouse. The subsequent growth of a teratocarcinoma, displaying the presence of tissues derived from all three germinal layers, provides functional evidence of the proliferation of a pluripotent stem cell by the adipose tissue-derived stromal layer.

### Example 3

### Gene Therapy Modifications

The following experimental outline describes an approach to convert adipose tissue-derived stromal cells into cells expressing factors, including but not limited to, leukemia inhibitory factor, IL-1 through IL-13, IL-15 through IL-17, IL-19 through IL-22, granulocyte macrophage colony stimulating factor (GM-CSF), granulocyte colony stimulating factor (G-CSF), macrophage colony stimulating factor (M-CSF), erythropoietin (Epo), thrombopoietin (Tpo), Flt3-ligand, BAFF (novel ligand of TNF family for B cell activating factor), artemin (a neurotrophic factor belonging to the GDNF family), bone morphogenic protein factors, epidermal growth factor (EGF), glial derived neurotrophic factor, lymphotactin, macrophage inflammatory proteins (alpha and beta), myostatin (also known as Growth Differentiation Factor-8), neurturin, nerve growth factors, platelet derived growth factors, placental growth factor, pleiotrophin, stem cell factor, stem cell growth factors, transforming growth factors, tumor necrosis factors, Vascular Endothelial Cell Growth Factors, and fibroblast growth factors including FGF-4 through FGF-10, FGF-16 through FGF-20, FGF-acidic or basic fibroblast growth factor, to improve their embryonic stem cell support capacity in a co-culture system. Measures of the embryonic stem cell function are also included.

Human adipose tissue-derived stromal cells are genetically engineered to express exogenous genes to enhance their ability to support the proliferation and maintenance of human embryonic stem cells in vitro. Cultures of primary human adipose tissue-derived stromal cells are transduced or transfected with appropriate vectors encoding the cDNAs for human leukemia inhibitory factor (LIF), human basic fibroblast growth factor (bFGF), epidermal growth factor (EGF) or a related cytokine or growth factor identified as supportive of embryonic stem proliferation.

The transduced or transfected human adipose-derived stromal cells are used to prepare a feeder layer as indicated under Examples 1 and 2 above. The presence of the genetically modified feeder layer is expected to reduce the need for the addition of exogenous growth factors in the co-culture maintenance of embryonic stem cells in vitro.

### Example 4

### Flow Cytometric Analysis of Human Adipose-Derived Stromal Cells

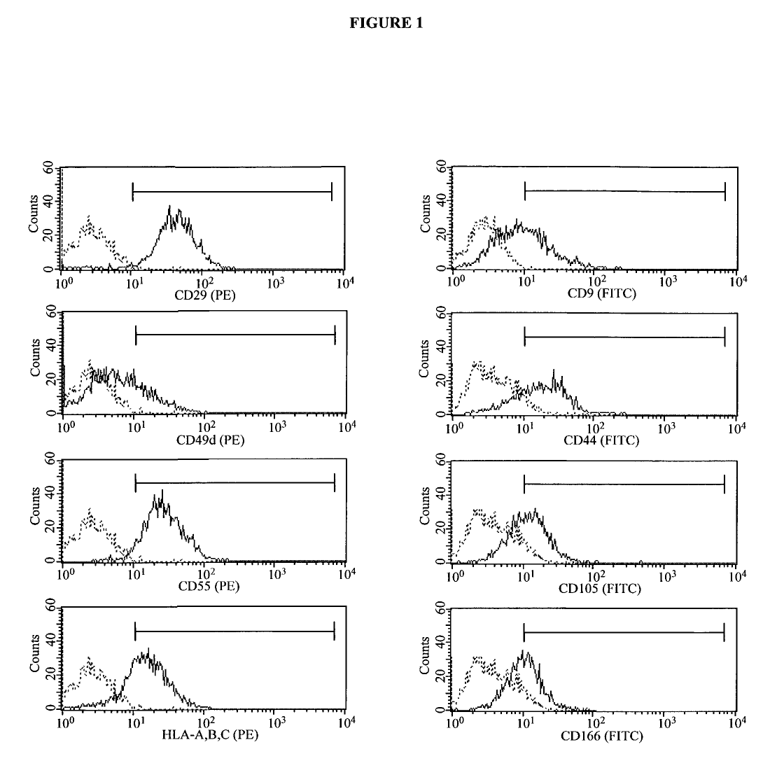

The adipose derived stromal cells express a number of adhesion and surface proteins; these are summarized in Table 1. Many of these proteins have the potential to serve a hematopoietic supportive function and all of them are shared in common by bone marrow stromal cells. Representative flow cytometric histograms are displayed also for CD9, CD29 (β1 integrin), CD44 (hyaluronate receptor), CD49_{d} (α4 integrin), CD55 (decay accelerating factor), and HLA-ABC (Class I histocompatibility antigen) (FIG. 1).

Undifferentiated human adipose derived stromal cells isolated from a single donor were stained with monoclonal antibodies against indicated antigens (solid line, right of each panel); isotype monoclonal control antibody (dotted line, left of each panel). Representative n=5 donors. Bar indicates fluorescent intensity >99% control.

TABLE 1
Adipose Derived Stromal Cell Surface Markers and Cytokines

| Cell Surface Markers | Cytokines |
|---|---|
| CD29-Integrin beta 1 | Interleukins 6, 7, 8, 11 |
| CD44-Hyaluronate Receptor | Macrophage-Colony Stimulating Factor |
| CD49d,e-Integrin alpha 4, 5 | GM-Colony Stimulating Factor |
| CD54-ICAM1 | Granulocyte-Colony Stimulating Factor |
| CD105-Endoglin | Leukemia Inhibitory Factor |
| CD106-VCAM-1 | Stem Cell Factor |
| CD166-ALCAM | Bone Morphogenetic Protein |

### Example 5

### PCR Analysis of Lipopolysaccharide (LPS) Induction of Cytokine mRNA

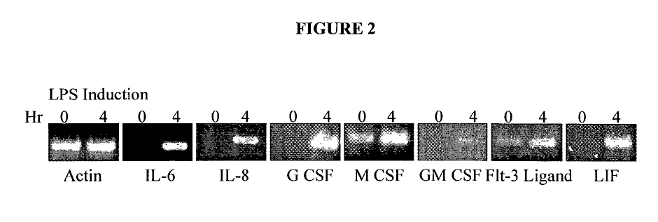

Adipose derived stromal cells were induced with 100 ng/ml LPS for 0 or 4 hours and harvested for total RNA. Reverse transcribed cDNAs were amplified with specific primer sets for interleukins 6 and 8, granulocyte-, macrophage-, and granulocyte/macrophage-colony stimulating factors, flt-3 ligand, and leukemia inhibitory factor (FIG. 2). Actin signal served as a control for equivalent cDNA levels in each reaction. PCR products were sequence confirmed.

The PCR results were confirmed at the protein level by ELISA assay (Table 2). As shown, the stromal cells significantly increase their secretion of IL-6, IL-7, IL-8, M-CSF, GM-CSF and TNFα within 24 hours following induction with LPS.

The cytokine expression profile of human adipose derived stromal cells from multiple donors (Table 2) was determined. In these experiments, confluent, quiescent adipose derived stromal cell cultures were induced with lipopolysaccharide (LPS, 100 ng/ml) and conditioned medium and total RNA were harvested after periods of 1 to 24 hours. In common with both murine and human bone marrow-derived stromal cells, adipose derived stromal cells expressed the following cytokine mRNAs: interleukins 6, 7, 8, and 11 (IL-6,-7,-8,-11), leukemia inhibitory factor (LIF), macrophage-colony stimulating factor (M-CSF), granulocyte-macrophage-colony stimulating factor (GM-CSF), granulocyte-colony stimulating factor (G-CSF), flt-3 ligand, stem cell factor, tumor necrosis factor α (TNFα) and bone morphogenetic proteins 2 and 4 t (BMP-2, -4) (FIG. 2).

TABLE 2
Lipopolysaccharide Induction of Secreted Cytokines (ELISA, pg/ml)

| Time LPS | 0 Hr | 1 Hr | 2 Hr | 4 Hr | 8 Hr | 24 Hr |
|---|---|---|---|---|---|---|
| GM-CSF* | 1 ± 1 | 1 ± 0 | 3 ± 1 | 7 ± 2 | 17 ± 3 | 76* ± 28 |
| M-CSF* | 4 ± 3 | 76 ± 14 | 161 ± 29 | 304 ± 62 | 512 ± 98 | 977* ± 285 |
| TNFα* | 0 ± 0 | 0 ± 0 | 5 ± 8 | 38 ± 33 | 112* ± 82 | 30 ± 22 |
| IL-1 α/β | N.D | N.D | N.D. | N.D. | N.D. | N.D. |
| IL-6* | 1 ± 1 | 287 ± 73 | 674 ± 51 | 2649 ± 495 | 6083 ± 956 | 9204* ± 2676 |
| IL-7* | 0.4 ± 0.2 | 0.4 ± 0.2 | 0.3 ± 0.3 | 0.3 ± 0.3 | 0.9 ± 0.2 | 3.4* ± 0.7 |
| IL-8* | 0 ± 0 | 88 ± 42 | 225 ± 82 | 1343 ± 224 | 4924 ± 1046 | 9710* ± 2438 |
| IL-11 | 2 ± 2 | 2 ± 1 | 13 ± 6 | 14 ± 6 | 16 ± 6 | 19 ± 8 |
| IL-12 | N.D | N.D | N.D. | N.D. | N.D. | N.D. |

(Values in Table 2 are the mean ± S.E.M. from n = 5 to 8 stromal cells donors. ELISAs were performed with undiluted, 1:25 or 1:125 diluted conditioned medium after the indicated exposure time to 100 ng lipopolysaccharide per ml of medium. The IL-7 ELISA is linear between 0.16 to 10 pg/ml. Astericks indicate *p < 0.05 between 8 or 24 hour and 0 hour time points based on one-way ANOVA. Abbreviation: N.D., not detectable.)

### Example 6

### Fold-Expansion

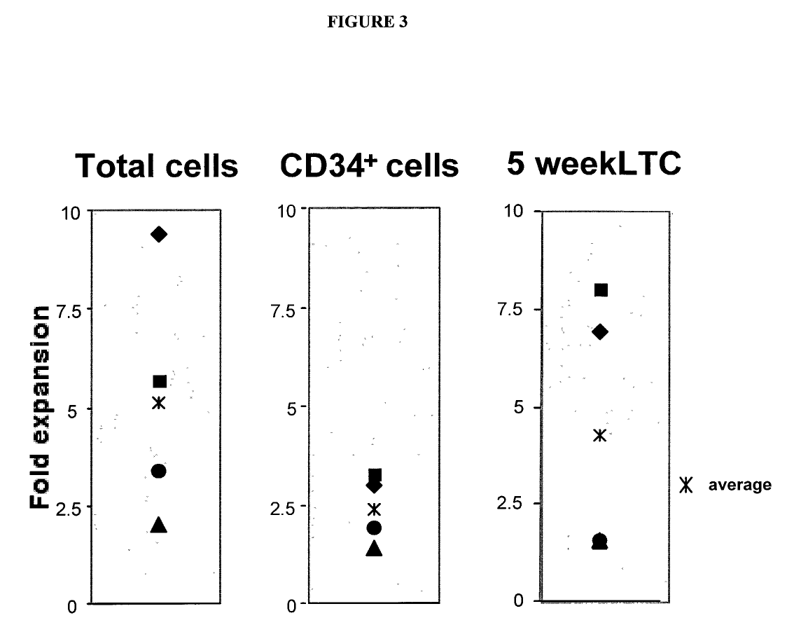

The ability of human adipose-derived stromal cells to support the proliferation and differentiation of human umbilical cord blood CD34⁺ hematopoietic progenitor cells in vitro was examined. Confluent cultures of adipose-derived stromal cells were established in 24 well plates (6×10⁴ cells per well). Umbilical cord blood specimens were depleted of contaminating erythrocytes by treatment with hetastarch and of contaminating granulocytes by Ficoll density centrifugation. The remaining UCB mononuclear cells were lineage depleted according to the StemSep™ (StemCells, Vancouver, BC) protocol; this relies on immunomagnetic negative cell selection using a cocktail of antibodies directed against CD2, CD3, CD14, CD16, CD19, CD24, CD56, CD66b, and glycophorin A. In the last purification step, the lin⁻UCB cells were stained with CD34 antibodies and sorted by flow cytometry. Up to 10,000 of the final CD34⁺ UCB cells have been co-cultured in individual wells with a confluent adipose-derived stromal cell layer. Cultures were maintained in the absence of exogenous cytokines for periods of 12-days, 3 weeks, or 6 weeks. At the end of these periods, individual wells were harvested by trypsin/EDTA digestion and analyzed by flow cytometry using a combination of the following antibody combinations (fluorescent tags indicated in parentheses): CD45 (FITC), CD34 (APC), and either CD7, CD10, or CD38 (PE). FIG. 3 demonstrates that hematopoietic cells from 12 day adipose stroma co-cultures were examined for total cell expansion (left panel), CD34+ cell expansion (middle panel) or seeded on MS5 cells for 5 weeks and the expansion of myeloid long term culture initiating (LTC) cells.

In the absence of exogenous cytokines, adipose-derived stromal cells supported a 5.1-fold expansion of total hematopoietic cell numbers (average, n=4 stromal donors, n=2 UCB donors; range 2-9.4) (FIG. 3). This corresponded to a 2.4-fold expansion of the CD34⁺ UCB cell population (average, n=4 stromal donors, n=2 UCB donors; range 1.4-3.3) (FIG. 3).

Modifications and other embodiments of the invention will be apparent to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. A composition comprising an isolated stromal cell capable of supporting the in vitro proliferation and maintenance of stem cells in combination with a stem cell.

2. The composition of claim 1, wherein the stromal cell is human.

3. The composition of claim 1, wherein exogenous genetic material has been introduced into the stromal cell.

4. The composition of claim 1, wherein the stromal cell secretes a protein.

5. The composition of claim 1, wherein the protein secreted is a growth factor, cytokine, or any protein promoting the proliferation of the stem cell.

6. The composition of claim 1, wherein the stromal cell is irradiated.

7. The composition of claim 1, wherein the stromal cell is derived from adipose tissue.

8. The composition of claim 1, wherein the stromal cell is derived from bone marrow.

9. The composition of claim 1, wherein the stromal cell is derived from ligamentous tissue or tendon.

10. The composition of claim 1, wherein the stromal cell is derived from skeletal muscle.

11. The composition of claim 1, wherein the stromal cell is derived from smooth muscle.

12. The composition of claim 1, wherein the stromal cell is derived from bone.

13. The composition of claim 1, wherein the stromal cell is derived from cartilage.

14. The composition of claim 1, wherein the stromal cell is derived from connective tissue.

15. The composition of claim 1, wherein the stromal cell is derived from peripheral blood.

16. The composition of claim 1, wherein the stromal cell is derived from skin.

17. The composition of claim 1, wherein the stromal cell is derived from umbilical cord blood.

18. The composition of claim 1, wherein the stromal cell is derived from placenta.

19. The composition of claim 1, wherein the stem cell is embryonic in origin.

20. The composition of claim 1, wherein the stem cell is adult in origin.

21. The composition of claim 1, wherein the stem cell expresses telomerase.

22. The composition of claim 1, wherein the stem cell is selected from the group comprising neuronal stem cell, liver stem cell, hematopoietic stem cell, umbilical cord blood stem cell, epidermal stem cell, gastrointestinal stem cell, endothelial stem cell, muscle stem cell, mesenchymal stem cell and pancreatic stem cell.

23. The composition of claim 1, wherein the stem cell remains undifferentiated.

24. A method for the growth and maintenance of cultured stem cells comprising:
i) isolating tissue-derived stromal cells; and
ii) culturing the stromal cells in culture media with stem cells.

25. The method of claim 24 further comprising culture supplemented with growth factors, cytokines and chemokines.

26. The method of claim 26, wherein the growth factors, cytokines, and chemokines are selected from the group consisting of: leukemia inhibitory factor, IL-1 through IL-13, IL-15 through IL-17, IL-19 through IL-22, granulocyte macrophage colony stimulating factor (GM-CSF), granulocyte colony stimulating factor (G-CSF), macrophage colony stimulating factor (M-CSF), erythropoietin (Epo), thrombopoietin (Tpo), Flt3-ligand, B cell activating factor, artemin, bone morphogenic protein factors, epidermal growth factor (EGF), glial derived neurotrophic factor, lymphotactin, macrophage inflammatory proteins, myostatin, neurturin, nerve growth factors, platelet derived growth factors, placental growth factor, pleiotrophin, stem cell factor, stem cell growth factors, transforming growth factors, tumor necrosis factors, Vascular Endothelial Cell Growth Factors, and fibroblast growth factors, FGF-acidic and basic fibroblast growth factor.

27. The method of claim 25, wherein the growth factors, cytokines and chemokines promote the differentiation of the stem cells.

28. The method of claim 25, wherein the growth factors, cytokines and chemokines inhibit the differentiation of the stem cells.

29. The method of any of claims 24-28, wherein the isolated stromal cells are irradiated prior to culturing with the stem cells.

30. The method of any of claims 24-29, wherein the stem cells are of embryonic origin.

31. The method of any of claims 24-29, wherein the stem cells are of adult origin.

32. The method of any of claims 24-29, wherein the stem cells are selected from the group comprising neuronal stem cells, liver stem cells, hematopoietic stem cells, umbilical cord blood stem cells, epidermal stem cells, gastrointestinal stem cells, endothelial stem cells, muscle stem cells, mesenchymal stem cells and pancreatic stem cells.

33. The method of claim 24-29, wherein the stromal cells are isolated from a source comprising adipose tissue, bone marrow, ligamentous tissue or tendon, skeletal muscle, smooth muscle, bone, cartilage, connective tissue, peripheral blood, skin, umbilical cord blood, and placenta.

34. The method of any of claims 24-33, wherein the isolated stromal cells are genetically engineered to express a growth factor, cytokine or chemokine.

35. The method of claim 34, wherein the growth factor, cytokine, and chemokine are selected from the group consisting of: leukemia inhibitory factor, IL-=b 1 through IL-13, IL-15 through IL-17, IL-19 through IL-22, granulocyte macrophage colony stimulating factor (GM-CSF), granulocyte colony stimulating factor (G-CSF), macrophage colony stimulating factor (M-CSF), erythropoietin (Epo), thrombopoietin (Tpo), Flt3-ligand, B cell activating factor, artemin, bone morphogenic protein factors, epidermal growth factor (EGF), glial derived neurotrophic factor, lymphotactin, macrophage inflammatory proteins, myostatin, neurturin, nerve growth factors, platelet derived growth factors, placental growth factor, pleiotrophin, stem cell factor, stem cell growth factors, transforming growth factors, tumor necrosis factors, Vascular Endothelial Cell Growth Factors, and fibroblast growth factors, FGF-acidic and basic fibroblast growth factor.

36. The method of claim 32, wherein the stem cells are maintained in an undifferentiated state.

37. The method of claim 32, wherein the stem cells are differentiated in culture.
